(19) United States
(12) Patent Application Publication
(10) Pub. No.: US 2016/0199028 A1
(43) Pub. Date: Jul. 14, 2016

(54) WIRELESS ULTRASONIC PROBE AND ULTRASONIC APPARATUS HAVING THE SAME

(71) Applicant: SAMSUNG MEDISON CO., LTD., Gangwan-do (KR)

(72) Inventors: Nam Du JEON, Seoul (KR); Han Eol KIM, Seoul (KR); Dong Gyu HYUN, Gwangju-si (KR)

(21) Appl. No.: 14/964,314

(22) Filed: Dec. 9, 2015

(30) Foreign Application Priority Data
Jan. 13, 2015 (KR) 10-2015-0006282

(51) Int. Cl. A61B 8/00; G06K 7/10; A61B 8/08

(52) U.S. Cl. CPC A61B 8/4472; A61B 8/56; A61B 8/54; A61B 8/467; A61B 8/5207; G06K 7/10366; A61B 2560/0214

(57) ABSTRACT

Disclosed is a ultrasonic apparatus including a wireless ultrasonic probe at which a RFID antenna receiving a RFID signal from outside and a ultrasonic apparatus having the same. The wireless ultrasonic probe includes a housing and a RFID antenna provided inside the housing to receive a RFID signal.

### CROSS-REFERENCE TO RELATED APPLICATION

This application claims the benefit of Korean Patent Application No. 10-2015-0006282, filed on Jan. 13, 2015, in the Korean Intellectual Property Office, the disclosure of which is incorporated herein by reference.

### BACKGROUND

1. Field

Embodiments of the present disclosure relate to a wireless ultrasonic probe performing communication with a main body and an ultrasonic apparatus having the same.

2. Description of the Related Art

Ultrasonic apparatuses operate to transmit ultrasonic waves through the surface of a subject to a target site inside a body and receive echo ultrasonic waves reflected from the target site to obtain a cross-sectional image of a soft tissue or bloodstream by using information about the echo ultrasonic waves in a non-invasive manner.

A ultrasonic probe forming the ultrasonic apparatus transmits ultrasonic waves to a subject and receives echo-ultrasonic waves reflected from the subject. In detail, the ultrasonic probe operates to convert an electric signal to ultrasonic waves, transmit the converted ultrasonic waves to a subject, receive echo-ultrasonic waves reflected from the subject, convert the received echo-ultrasonic waves into an echo-ultrasonic signal, and transmit the echo-ultrasonic signal to a main body of the ultrasonic apparatus.

In the recent years, numerous studies have been conducted on a wireless ultrasonic probe that is connected to a main body in a wireless scheme to receive a ultrasonic signal and transmit an echo-ultrasonic signal.

### SUMMARY

Therefore, it is an aspect of the present disclosure to provide a wireless ultrasonic probe provided with a RFID antenna that receives a RFID signal from outside, and an ultrasonic apparatus having the same.

In accordance with an embodiment of the present disclosure, a wireless ultrasonic probe includes a housing and a radio frequency identification (RFID) antenna. The RFID antenna may be provided inside the housing to receive a RFID signal.

The wireless ultrasonic probe may further include a piezoelectric member and a wireless communicator. The piezoelectric member may be configured to transmit ultrasonic waves at an object and receive echo-ultrasonic waves reflected from the object. The wireless communicator may be configured to transmit an echo-signal corresponding to the echo-ultrasonic waves.

The RFID antenna may include a substrate and an antenna pattern. The substrate may include a first surface and a second surface being opposite and spaced away from the first surface. The antenna pattern may be formed on the first surface or the second surface.

The RFID antenna may be provided such that a first direction perpendicular to the first surface and the second surface of the substrate is different from a second direction corresponding to an irradiation direction of the ultrasonic waves.

The RFID antenna may be provided inside the housing such that an angle formed between the first direction and the second direction is 30 degrees or above.

The wireless ultrasonic probe may further include a controller configured to generate a control signal to control the wireless ultrasonic probe.

The wireless ultrasonic probe may further include an input tag provided with a plurality of RFIDs that is selectable by a user.

The RFID antenna may sense at least one RFID tag selected by the user in the input tag.

The controller may generate a control signal corresponding to the sensed at least one RFID tag.

The RFID antenna may receive a RFID signal including identification information about an external device from a RFID antenna of the external device.

The controller may generate a control signal communicating with the external device corresponding to the received identification information.

The wireless communicator may transmit the echo-signal to the external device according to the control signal.

The RFID antenna may receive a RFID signal including setting information about the external device or an echo-signal previously stored in the external device from the external device according to the control signal.

The wireless ultrasonic probe may further include a shielding film, a printed circuit board, and a battery. The shielding film may be provided inside the housing to protect the inside of the housing from noise. The PCB may be configured to provide a path of a signal inside the housing. The battery may be configured to supply power to the wireless ultrasonic probe. The RFID antenna may be provided on at least one of the shield film, the PCB substrate, the battery and the inside of the housing.

In accordance with another embodiment of the present disclosure, a ultrasonic apparatus includes a wireless ultrasonic probe and a main body. The wireless ultrasonic probe may be configured to transmit ultrasonic waves at an object, receive echo-ultrasonic waves reflected from the object, and transmit an echo-signal corresponding to the receive echo-ultrasonic waves in a wireless scheme. The main body may be configured to generate an ultrasonic image based on the echo signal received from the wireless ultrasonic probe. The wireless ultrasonic probe may include a RFID antenna provided inside a housing of the wireless ultrasonic probe to receive a RFID signal.

The RFID antenna may include a substrate and an antenna. The substrate may include a first surface and a second surface being opposite and spaced away from the first surface. The antenna pattern may be formed on the first surface or the second surface.

The RFID antenna may be provided such that a first direction perpendicular to the first surface and the second surface of the substrate is different from a second direction corresponding to an irradiation direction of the ultrasonic waves.

The RFID antenna may be provided inside the housing such that an angle formed between the first direction and the second direction is 30 degrees or above.

The wireless ultrasonic probe may further include a controller configured to generate a control signal to control the wireless ultrasonic probe.

The ultrasonic apparatus may further include an input tag provided with a plurality of RFID that is selectable by a user.

The RFID antenna may sense at least one RFID tag selected by the user in the input tag.

The controller may generate a control signal corresponding to the sensed at least one RFID tag.

The RFID antenna may receive a RFID signal including identification information about the main body from the main body.

The controller may generate a control signal communicating with the main body corresponding to the received identification information.

The wireless ultrasonic probe may transmit the echo-signal to the main body device according to the control signal.

As is apparent from the above, the wireless ultrasonic probe according to the present disclosure and a ultrasonic apparatus having the same can simplify a pairing process with respect to a main body by identifying the main body based on a received RFID signal. In addition, the wireless ultrasonic probe can easily perform pairing with an external device other than the main body.

In addition, the wireless ultrasonic probe according to the present disclosure and a ultrasonic apparatus having the same can communicate data including setting information about the wireless ultrasonic probe by allowing a RFID signal to be transmitted between a plurality of wireless ultrasonic probes.

### BRIEF DESCRIPTION OF THE DRAWINGS

These and/or other aspects of the disclosure will become apparent and more readily appreciated from the following description of the embodiments, taken in conjunction with the accompanying drawings of which:

FIG. 1 is a view illustrating an external appearance of a ultrasonic apparatus according to an embodiment of the present disclosure;

FIG. 2 is a control block diagram of a ultrasonic apparatus according to an embodiment of the present disclosure;

FIG. 3 is a view illustrating an external appearance of a wireless ultrasonic probe according to an embodiment of the present disclosure, and an external appearance of a RFID antenna included in a wireless ultrasonic probe according to an embodiment of the present disclosure;

FIG. 4 is a view illustrating methods of forming RFID antennas at a wireless ultrasonic probe according to various embodiments of the present disclosure;

FIGS. 5A and 5B are views illustrating a paring method in an ultrasonic apparatus according to an embodiment of the present disclosure;

FIGS. 6A and 6B are views illustrating a communication method of a wireless ultrasonic probe and an input tag according to an embodiment of the present disclosure;

FIG. 7 is a view illustrating a communication method in which a wireless ultrasonic probe according to an embodiment of the present disclosure performs communication another wireless ultrasonic probe; and

FIG. 8 is a view illustrating a communication method in which a wireless ultrasonic probe according to an embodiment of the present disclosure performs communication with another external device.

### DETAILED DESCRIPTION

Reference will now be made in detail to the embodiments of the present disclosure, examples of which are illustrated in the accompanying drawings, wherein like reference numerals refer to like elements throughout.

Hereinafter, a wireless ultrasonic probe and a ultrasonic apparatus having the same will be described with reference to the accompanying drawings in detail.

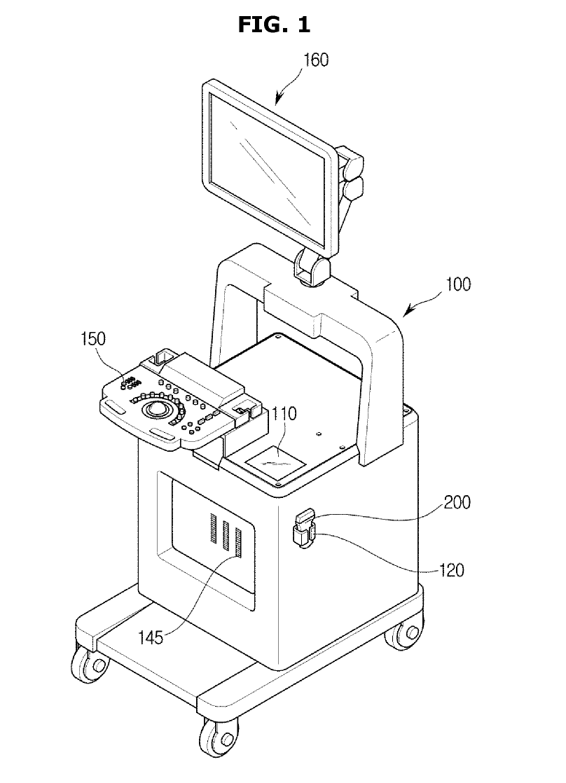

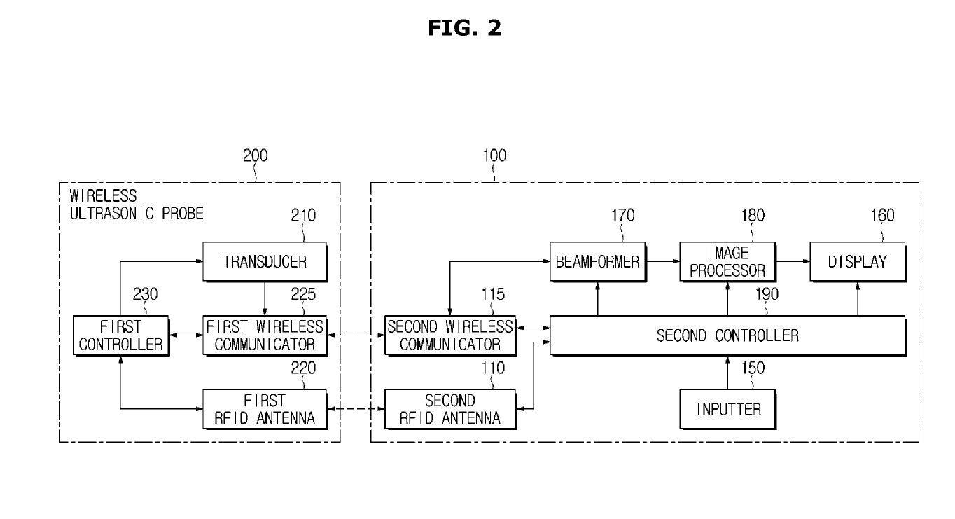

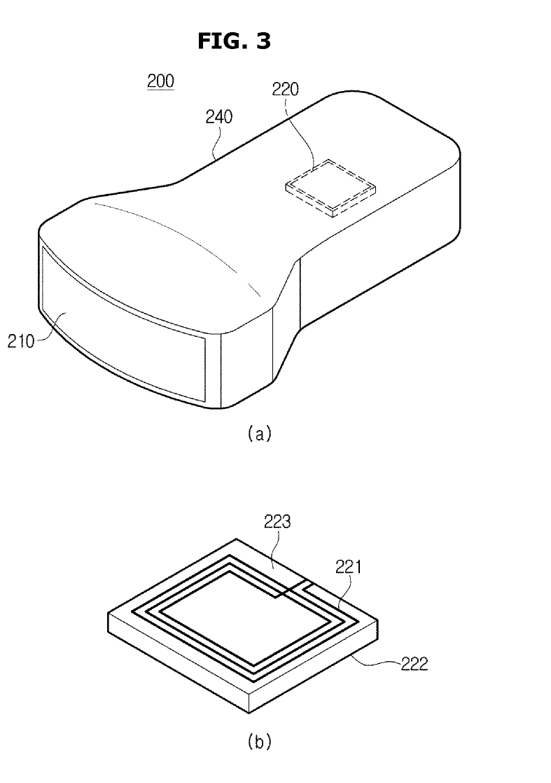

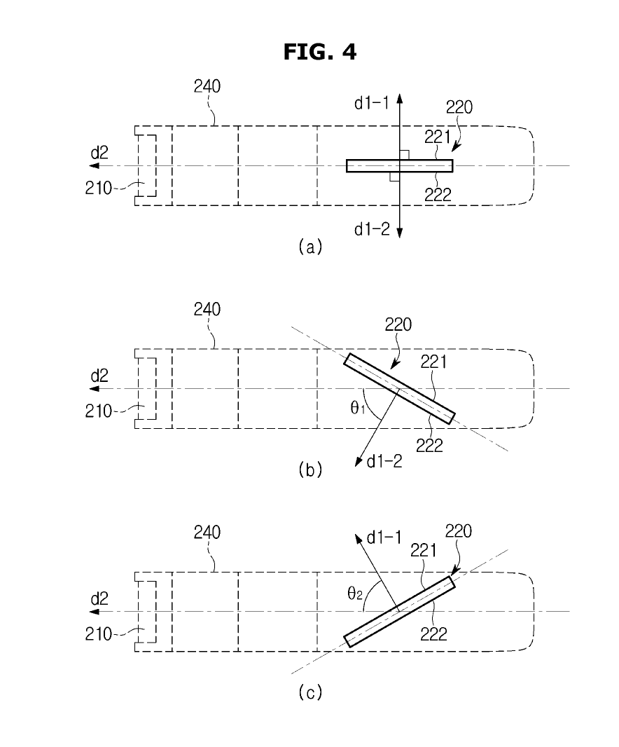

FIG. 1 is a view illustrating an external appearance of a ultrasonic apparatus according to an embodiment of the present disclosure, FIG. 2 is a control block diagram of a ultrasonic apparatus according to an embodiment of the present disclosure, FIG. 3 is a view illustrating an external appearance of a ultrasonic probe according to an embodiment of the present disclosure, and an external appearance of a RFID antenna included in a ultrasonic probe according to an embodiment of the present disclosure, and FIG. 4 is a view illustrating methods of forming RFID antennas at a ultrasonic probe according to various embodiments of the present disclosure.

Referring to FIG. 1, a ultrasonic apparatus includes a main body 100 and a wireless ultrasonic probe 200 connected to the main body 100 in a wireless scheme.

The main body 100 may receive an echo-signal in the form of an electric signal corresponding to echo-ultrasonic waves received by the wireless ultrasonic probe 200, and generated a ultrasonic image based on the echo-signal.

To this end, the main body 100 may include a second wireless communicator 115 receiving an echo-signal from the wireless ultrasonic probe 200, a beamformer 170 applying time delays to the received echo-signals and receiving the time delayed echo-signal, an image processor 180 generating an ultrasonic image based on the received echo-signal, a display 160 providing the generated ultrasonic image in a visual form, an inputter 150 receiving a control command from a user, and a second controller 190 controlling each component of the main body 100 according to the received control command or an internal operation.

The second wireless communicator 115 may perform a wireless communication with the wireless ultrasonic probe 200. In detail, the second wireless communicator 115 transmits an ultrasonic signal to generate ultrasonic waves to the wireless ultrasonic probe 200, and receive an echo-signal in the form of an electric signal corresponding to echo-ultrasonic waves from the wireless ultrasonic probe 200.

To this end, the second wireless communicator 115 may communicate with the wireless ultrasonic probe 200 by accessing the same network to which the wireless ultrasonic probe 200 makes an access. Alternatively, the second wireless communicator 115 may communicate with the wireless ultrasonic probe 200 in the near field communication (NFC) method, such as Wireless LAN, Wi-Fi, Bluetooth, Zigbee, Wi-Fi Direct (WFD), Ultra Wideband (UWB), Infrared Data Association (IrDA) and Bluetooth Low Energy (BLE).

The second wireless communicator 115 performs communication only with the wireless ultrasonic probe 200 having been finished with pairing. Details thereof will be described later.

The beamformer 170 may perform beamforming to receive ultrasonic waves. When ultrasonic waves are transmitted to a certain position of the object ob, there is time difference between the ultrasonic waves arriving at the certain position, or when echo-ultrasonic waves are received from a certain position of an object ob to the wireless ultrasonic probe 200, there is time difference between the echo-ultrasonic waves arriving at the ultrasonic probe 200. Accordingly, the time difference is compensated through the beamforming.

The beamformer 170 includes a pulser configured to generate a ultrasonic signal, which is a pulse or an alternating current signal, a transmission beamformer configured to delay a ultrasonic signal to arrange ultrasonic waves transmitted at a certain position of an object, and a reception beamformer configured to delay an echo signal to arrange echo-ultrasonic waves reflected from a certain position of the object.

The transmission beamformer delays a ultrasonic signal generated from the pulser, thereby performing transmission beamforming on the ultrasonic signal. The beamformed ultrasonic signal may be transmitted to the wireless ultrasonic probe 200 by the second wireless communicator 115.

In addition, the reception beamformer delays an echo signal received from the wireless ultrasonic probe 200, thereby performing reception beamforming.

The beamformer 170 may be implemented by adopting any one of generally known beamforming methods, and a plurality of beamforming methods may be used, or selectively used.

The image processor 180 may generate a ultrasonic image by processing the beamformed echo-signal. The image processor 180 may process the echo-signal according to any one of generally known image processing methods.

For example, the image processor 180 may perform a scan conversion with respect to the beamformed echo-signal. The scan conversion may represent a process of storing an echo signal in a memory of the image processor 180 based on coordinates of an ultrasonic image desired to display. In this manner, the image processor 180 may convert echo-signals into a frame image of an ultrasonic image.

Before storing the echo-signals in the memory, the image processor 180 may perform a pre-processing on the echo-signals, for example, Edge enhancement, Pixel Interpolation, Persistence, Panoramic Imaging, Spatial Compounding, and 3D Acquisition.

In addition, after storing the echo-signals in the memory, the image processor 180 may perform a post-processing on the converted ultrasonic image. In this case, the post-processing may include all processes performed to display the echo-signal fetched from the memory.

The display 160 may display an ultrasonic image having been subject to the post-processing by the image processor 180. A user may visually check the ultrasonic image about an inside of the object provided through the display 160, inspecting a patient.

In addition, the display 160 may display various user interfaces (Uls) related to controlling the ultrasonic apparatus. A user may check the UI provided through the display 160, and input a command to control the ultrasonic apparatus or each component of the ultrasonic apparatus.

The display 160 may be implemented using one of a cathode ray tube (CRT) and a liquid crystal display (LCD). In addition, the display 160 may provide a three-dimensional image as well as a two-dimensional image.

The second controller 190 may control the beamformer 170, the image processor 180, the second wireless communicator 115 and/or the display 160 to control the overall operation of the ultrasonic apparatus. In addition, the inputter 150 may control each component of the ultrasonic apparatus according to a control command input by a user.

For example, the second controller 190 may control a method of beamforming in the beamformer 170, a method of generating a ultrasonic image in the image processor 180, and a method of displaying a ultrasonic image in the display 160. In addition, the second controller 190 may perform pairing with the wireless ultrasonic probe 200 corresponding to received identification information.

To this end, the second controller 190 generates a control signal corresponding a control command, and transmits a desired component to be controlled. The component of the ultrasonic apparatus having received the control signal may be controlled according to the control signal.

The wireless ultrasonic probe 200 at the time of being kept is coupled to a holder 120 provided at the main body 100, and at the time of being used by a user, is separated from the holder 120.

The wireless ultrasonic probe 200 transmits ultrasonic waves at an object based on a ultrasonic signal received from the main body 100, receives echo ultrasonic waves reflected from the object, and sends the main body 100 an echo-signal in the form of an electric signal corresponding to the received echo-ultrasonic waves.

In detail, the wireless ultrasonic probe 200 includes a housing 240, a transducer 210 provided on one surface of the housing 240 to transmit ultrasonic waves at an object and receive echo-ultrasonic waves reflected from the object, a first wireless communicator 225 transmitting an echo-signal corresponding to the echo-ultrasonic waves in a wireless scheme and a first controller 230 configured to control each component of the wireless ultrasonic probe 200.

The first wireless communicator 225 may perform a wireless communication with the main body 100. In detail, the first wireless communicator 225 receives an ultrasonic signal to generate ultrasonic waves from the main body 100, and transmits an echo-signal in the form of an electric signal corresponding to echo-ultrasonic waves to the main body 100.

To this end, the first wireless communicator 225 may communicate with the main body 100 by accessing the same network to which the main body 100 makes an access. Alternatively, the first wireless communicator 225 may communicate with the main body 100 according to near field communication (NFC) method. The first wireless communicator 225 may adopt the same wireless communication method as that used by the second wireless communicator 115.

The first wireless communicator 225 performs communication only with the main body 100 having been finished with pairing.

The transducer 210 may generate ultrasonic waves by vibrating according to a ultrasonic signal received from the main body 100. In this case, the transducer 210 receives a ultrasonic signal having been subject to transmission beamforming, so that the generated ultrasonic waves are received at a certain position of the object.

In addition, the transducer 210 may receive echo-ultrasonic waves reflected from a certain position of the object. As a result, the transducer 210 vibrates at a frequency corresponding to a frequency of the received echo-ultrasonic waves, and at the same time, generates an alternating current having a frequency corresponding to the vibration frequency, that is, an echo-signal.

To this end, the transducer 210 may include a piezoelectric member 210 disposed on one surface of the housing 240. For example, the piezoelectric member may be arranged in one direction on a surface of the housing 240, or may have a two dimensional arrangement.

The first controller 230 may control the transducer 210 and the first wireless communicator 225 in order to control the overall operation of the wireless ultrasonic probe 200. To this end, the first controller 230 generates a control signal and transmits the control signal to a desired component to be controlled. The component of the wireless ultrasonic probe 200 having received the control signal may be operated under the control according to the control signal.

Meanwhile, the wireless ultrasonic probe 200 may perform pairing to communicate with the main body 100. Pairing represents an authentication procedure performed between devices to communicate with each other. In detail, the main body 100 may transmit identification information about the main body 100 to the wireless ultrasonic probe 200, and the wireless ultrasonic probe 200 may transmit identification information about the wireless ultrasonic probe 200 to the main body 100. The main body 100 is enabled to perform communication with the wireless ultrasonic probe 200 corresponding to the received identification information, and the wireless ultrasonic probe 200 is enabled to perform communication with the main body 100 corresponding to the received identification information, thereby finishing pairing.

In this case, the wireless ultrasonic probe 200 and the main body 100 may transmit and receive identification information thereof between each other through a RFID signal. To this end, the wireless ultrasonic probe 200 may further include a first RFID antenna 220, and the main body 100 may further include a second RFID antenna 110.

The first RFID antenna 220 and the second RFID antenna 110 may transmit and receive various bands of RFID signals, and preferably, may use RFID signals having a frequency of 13.56 MHz. When communication is performed by using a RFID signal having such a frequency band, the wireless ultrasonic probe 200 may transmit and receive data between terminals in a near area of about 10 cm. When a RFID signal having a frequency of 13.56 MHz is used, the first RFID antenna 220 and the second RFID antenna 110 may be implemented using a near field communication (NFC) antenna.

Referring to (a) of FIG. 3, the wireless ultrasonic probe 200 may be provided with the first RFID antenna 220 in the housing 240. In this case, the first RFID antenna 220 may include a substrate including a first surface 221, a second surface 222 being opposite and spaced away from the first surface 221, and an antenna pattern 223 formed on the first surface 221 or the second surface 222.

The substrate may be provided in the form of a flexible printed circuit board (FPCB) so as to be easily mounted inside the housing 240 of the wireless ultrasonic probe 200.

The antenna pattern 223 may include a pattern 223 having a length corresponding to a frequency band of a RFID signal, and may be provided in the form of a loop antenna along a periphery of the first surface 221 or the second surface 222. The antenna pattern 223 transmits and receives a RFID signal, and a surface of the first RFID antenna 220 on which the antenna pattern 223 is formed has the highest reception rate of RFID signals.

Referring to (b) of FIG. 3, the antenna pattern 223 is formed on the first surface 221 of the substrate.

The first RFID antenna 220 may be installed inside the housing 240 of the wireless ultrasonic probe 200 in various methods. For example, the first RFID antenna 22 may be formed on a shielding film formed of metal inside the housing 240 of the wireless ultrasonic probe 200 to block noise. Alternatively, the first RFID antenna 220 may be attached to an inner surface of the housing 240, and may be installed on a battery for supplying power to the wireless ultrasonic probe 200 inside the housing 240.

In this case, the first RFID antenna 220 may be provided inside the housing 240 such that first directions d1-1 and d1-2 perpendicular to the first surface 331 and the second surface 222 are different from a second direction d2 corresponding to an irradiation direction of the ultrasonic waves.

FIG. 4 is a view illustrating methods of forming the RFID antennas 220 at a ultrasonic probe according to various embodiments of the present disclosure.

In (a) of FIG. 4, the first RFID antenna 220 is installed parallel to the second direction d2 in which ultrasonic waves are transmitted. That is, the first directions d1-1 and d1-2 perpendicular to the first surface 221 and the second surface 222 of the first RFID antenna 220 may be perpendicular to the second direction d2 in which ultrasonic waves are transmitted.

In addition, the first RFID antenna 220 may be installed to be inclined with respect to the second direction d2 in which ultrasonic waves are transmitted. Referring to (b) of FIG. 4, the first direction d1-2 perpendicular to the second surface 222 of the first RFID antenna 220 may form an angle of θ1 with respect to the second direction d2 in which ultrasonic waves are transmitted. In addition, referring to (c) of FIG. 4, the first direction d1-1 perpendicular to the first surface 221 of the first RFID antenna 220 may form an angle of θ2 with respect to the second direction d2 in which ultrasonic waves are transmitted.

In this case, when a direction perpendicular to the first surface 221 or the second surface 222 of the first RFID antenna 220 coincides with an irradiation direction of ultrasonic waves, that is, when θ1 or θ2 is 0, the first surface 221 or the second surface 222 of the first RFID antenna 220 faces an arrangement surface of the transducer 210. If the first surface 221 or the second surface 222 of the first RFID antenna 220 has an area larger than that of the arrangement of the transducer 210, the interior space of the housing 240 needs to be increased to accommodate the first RFID antenna 220. In this case, the volume of the wireless ultrasonic probe 200 is increased, causing complicating user's manipulation.

In addition, when θ1 or θ2 is 0, a surface of the first RFID antenna 220 which has the highest reception rate with respect to RFID signals is disposed to face a surface on which the transducer 210 is provided. As a result, the transducer 210 is disposed on a signal path between the first RFID antenna 220 and the second RFID antenna 110. If the first RFID antenna 220 is moved toward the second RFID antenna 110 of the main body 100 to transmit and receive RFID signals, the transducer 210 or ultrasonic waves transmitted from the transducer 210 may lower the reception rate of RFID signals received by the first RFID antenna 220.

Accordingly, the first RFID antenna 220 may be provided inside the housing 240 such that the first directions d-1 and d1-2 perpendicular to the first surface 221 and the second surface 222 are different from the second direction d2 in which ultrasonic waves are transmitted. For example, the first RFID antenna 220 may be provided inside the housing 240 such that the first direction d-1 and d1-2, perpendicular to the first surface 221 or the second surface 222, is disposed to be inclined by 30 degrees or above with respect to the second direction d2 in which ultrasonic waves are transmitted. For the volume of the housing 240 needs to be increased when the angle of θ1 or θ2 is below 30 degrees.

As a component corresponding to the first RFID antenna 220, the main body 100 may include the second RFID antenna 110. In detail, the main body 100 has the second RFID antenna 110 provided at a position in which RF signal communication is performed with the first RFID antenna 220. On FIG. 1, the second RFID antenna 110 is formed on the surface of the main body 100.

The wireless ultrasonic probe 200 and the main body 100 each check identification information about the other party, thereby performing a pairing.

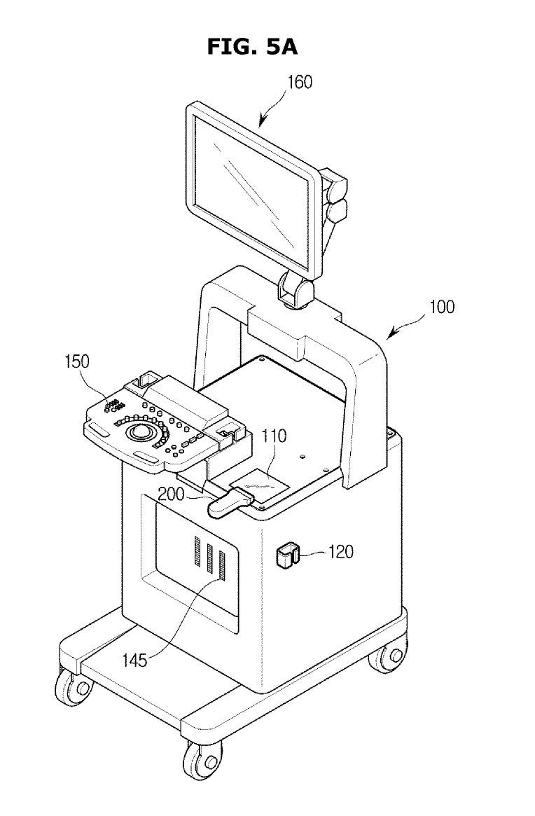

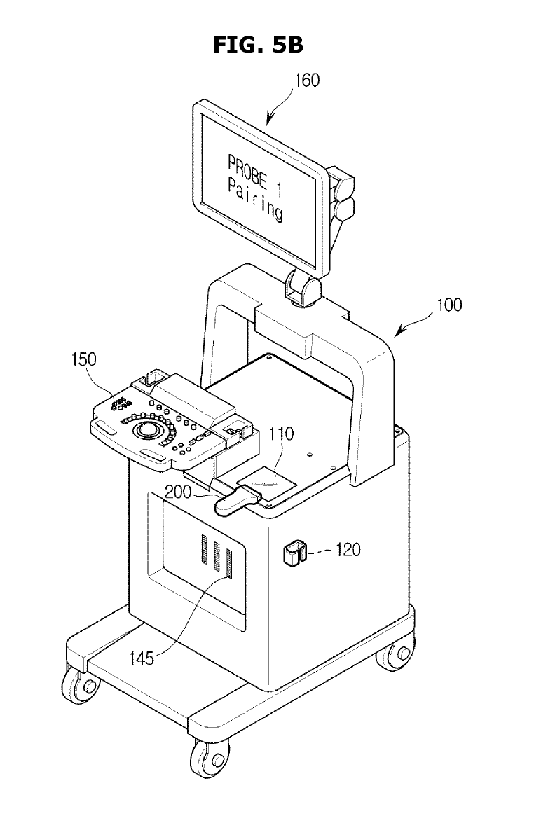

FIGS. 5A and 5B are views illustrating a paring method in an ultrasonic apparatus according to an embodiment of the present disclosure.

First, referring to FIG. 5A, the wireless ultrasonic probe 200 may be moved toward the main body 100 to be subject to pairing. In detail, the first RFID antenna 220 of the wireless ultrasonic probe 200 may be moved toward the second RFID antenna 110 of the main body 100.

As a result, the first RFID antenna 220 receives a RFID signal including identification information about the main body 100 from the second RFID antenna 110, and transmits the received RFID signal to the first controller 230. The first controller 230 may check the main body 100 corresponding to the identification information included in the received RFID signal.

In addition, the second RFID antenna 110 receives a RFID signal including identification information about the wireless ultrasonic probe 200 from the first RFID antenna 220, and transmits the received RFID signal to the second controller 190. The second controller 190 may check the wireless ultrasonic probe 200 corresponding to the identification information included in the received RFID signal.

As described above, the wireless ultrasonic probe 200 and the main body 100 transmit identification information thereof, receive identification information about a desired device to be pairing, and performing pairing.

Referring to FIG. 5B, when the pairing is complete, the display 160 may display a screen indicating that the pairing is complete.

When the pairing is complete, an echo-signal corresponding to echo-ultrasonic waves acquired by the transducer 210 of the wireless ultrasonic prove 200 is transmitted to the main body 100 that is paired with the wireless ultrasonic probe 200. In detail, the first controller 230 may control the first wireless communicator 225 to transmit an echo-signal converted in the transducer 210 to the second wireless communicator 115 of the main body 100. For example, the first controller 230 may send the first wireless communicator 225 a control signal allowing the first wireless communicator 225 to transmit an echo-signal to the second wireless communicator 115. The main body 100 may generate an ultrasonic image based on the echo-signal received through the second wireless communicator 115.

In addition, the main body 100 may send the wireless ultrasonic probe 200 a ultrasonic signal that has been subject to a transmission beamforming. In detail, the second controller 190 may control the second wireless communicator 115 to send the first wireless communicator 225 of the wireless ultrasonic probe 200 an ultrasonic signal having been subject to transmission beamforming in the beamformer 170. For example, the second controller 190 may send the second wireless communicator 115 a control signal allowing the second wireless communicator 115 to transmit a ultrasonic signal to the first wireless communicator 225. The wireless ultrasonic probe 200 may transmit ultrasonic waves at an object based on the ultrasonic signal received through the first wireless communicator 225.

Meanwhile, the wireless ultrasonic probe 200 may communicate with the main body 100 through the first RFID antenna 220, and also transmit and receive RFID signal to and from various external devices, respectively.

Hereinafter, a method of the wireless ultrasonic probe 200 communicating with various external devices by using the first RFID antenna 220 will be described.

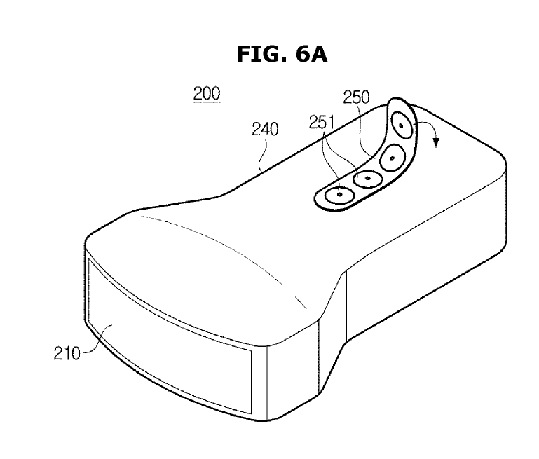

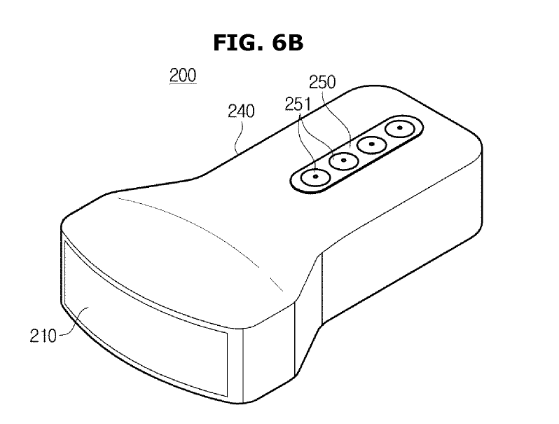

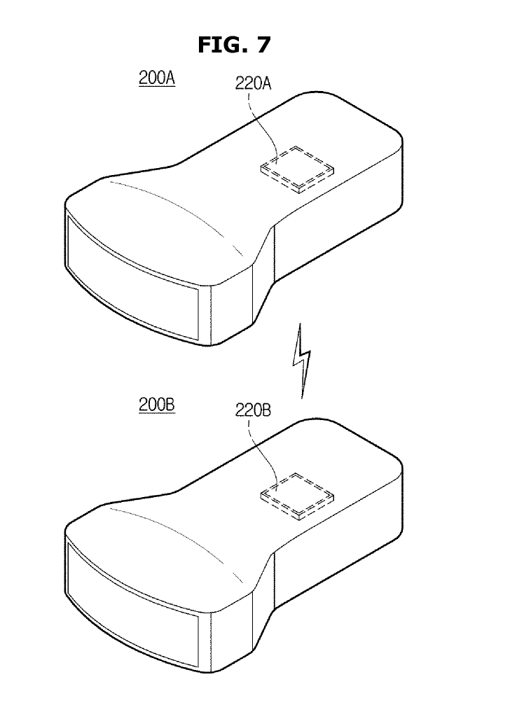

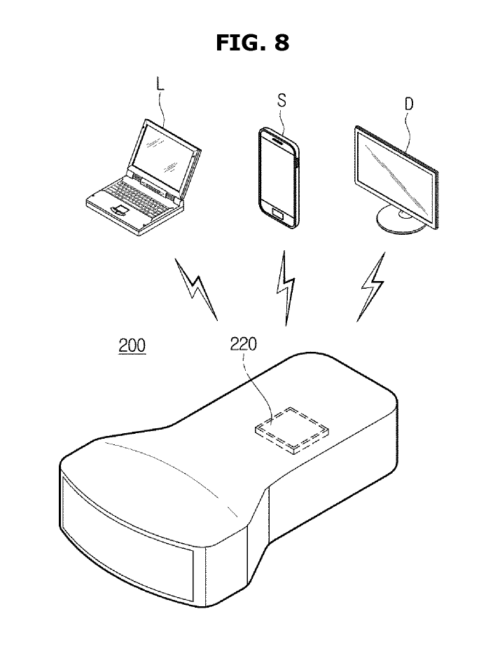

FIGS. 6A and 6B are views illustrating a communication method of a wireless ultrasonic probe and an input tag according to an embodiment of the present disclosure, and FIG. 7 is a view illustrating a communication method in which a wireless ultrasonic probe according to an embodiment of the present disclosure performs communication another wireless ultrasonic probe, FIG. 8 is a view illustrating a communication method in which a wireless ultrasonic probe according to an embodiment of the present disclosure performs communication with another external device.

The wireless ultrasonic probe 200 may further include an input tag 250 having a plurality of RFID tags 251 that are selectable by a user. Referring to FIG. 6A, the input tag 250 may be detachably provided at an outside of the housing 250.

FIG. 6B illustrates the input tag 250 attached to a surface of the housing 240 of the wireless ultrasonic probe 200. The input tag 250 may include a plurality of RFID tags 251, and a user may select at least one RFID tags from among the plurality of RFID tags 251, thereby inputting a control command.

In detail, the plurality of RFID tags 251 of the input tag 250 each may include different identification information. Before a user's selection is provided, the plurality of RFID tags 251 is not sensed by the first RFID antenna 220. If a user selects at least one RFID tag from among a plurality of RFID tags 220, the selected RFID tag 251 may be converted to be enabled to be sensed by the first RFID antenna 220.

As a result, the first RFID antenna 220 receives a RFID signal including identification information about the RFID tag 251 being enabled to be sensed, and transmits the identification information to the first controller 230. The first controller 230 checks the RFID tag 251 corresponding to the identification information included in the RFID signal, and generates a control signal corresponding to the control command input by the user.

For example, a user may press a RFID tag 251 corresponding to a power-on command from among the plurality of RFID tags 251. As a result, the pressed

RFID tag 251 is enabled to be sensed, and the first controller 230 may receive a RFID signal from the RFID tag 251 being enabled to be sensed. The first controller 230 checks identification information included in the received RFID signal, thereby checking that a user has input a power-on command. Finally, the first controller 230 generates a control command to turn on power of the wireless ultrasonic probe 200, thereby supplying power to the wireless ultrasonic probe 200.

In addition, the wireless ultrasonic probe 200 may exchange data with anther wireless ultrasonic probe 200 through RFID signals.

Referring to FIG. 7, a plurality of wireless ultrasonic probes 200A and 200B including first RFID antennas 220A and 220B may transmit and receive data therebetween. For example, the wireless ultrasonic probe 200A may receive predetermined setting information, which is set in the other wireless ultrasonic probe 200B, through a RFID signal. Alternatively, the wireless ultrasonic probe 200A may receive an echo-signal, which is previously acquired by the other wireless ultrasonic probe 200B, through a RFID signal.

In addition, prior to the data transmission/reception, the plurality of wireless ultrasonic probes 200A and 200B may perform pairing with each other by using the first RFID antennas 220A and 220B. That is, the wireless ultrasonic probes 200A and 200B each transmit a RFID signal including identification information thereabout through the first RFID antennas 220A and 220B, and receives a RFID signal including identification information about the other party.

The present disclosure is not limited thereto, and the wireless ultrasonic probe 200 may perform communication with various devices each including a RFID antenna.

Referring to FIG. 8, the wireless ultrasonic probe 200 may perform pairing with external devices such as a notebook computer (L), a smart phone (S) and/or a display device (D) each including a RFID antenna, through RFID signals. That is, the wireless ultrasonic probe 200 transmits a RFID signal including identification information thereabout to the external device through the first RFID antenna 220, and receives a RFID signal including identification information about the external device, thereby performing pairing.

When pairing is complete, the wireless ultrasonic probe 200 performs data communication with the external device. For example, the wireless ultrasonic probe 200 may transmit an echo-signal to the external device. In detail, the first controller 230 may send the first communicator a control signal allowing an echo-signal acquired by the transducer 210 to be transmitted to the external device. The first communicator may send an external device an echo signal according to the received control signal.

When an external device is provided with a display, the external device may display a ultrasonic image generated based on the received echo-signal. As a result, ultrasonic images may be provided to a user by using various devices.

Although a few embodiments of the present disclosure have been shown and described, it would be appreciated by those skilled in the art that changes may be made in these embodiments without departing from the principles and spirit of the disclosure, the scope of which is defined in the claims and their equivalents.

What is claimed is:

1. A wireless ultrasonic probe comprising:
a housing; and
a radio frequency identification (RFID) antenna provided inside the housing to receive a RFID signal.

2. The wireless ultrasonic probe of claim 1, further comprising:
a piezoelectric member configured to transmit ultrasonic waves at an object and receive echo-ultrasonic waves reflected from the object; and
a wireless communicator configured to transmit an echo-signal corresponding to the echo-ultrasonic waves.

3. The wireless ultrasonic probe of claim 1, wherein the RFID antenna comprises:
a substrate including a first surface and a second surface being opposite and spaced away from the first surface; and
an antenna pattern formed on the first surface or the second surface.

4. The wireless ultrasonic probe of claim 3, wherein the RFID antenna is provided such that a first direction perpendicular to the first surface and the second surface of the substrate is different from a second direction corresponding to an irradiation direction of the ultrasonic waves.

5. The wireless ultrasonic probe of claim 4, wherein the RFID antenna is provided inside the housing such that an angle formed between the first direction and the second direction is 30 degrees or above.

6. The wireless ultrasonic probe of claim 1, further comprising a controller configured to generate a control signal to control the wireless ultrasonic probe.

7. The wireless ultrasonic probe of claim 6, further comprising an input tag provided with a plurality of RFIDs that is selectable by a user.

8. The wireless ultrasonic probe of claim 7, wherein the RFID antenna senses at least one RFID tag selected by the user in the input tag.

9. The wireless ultrasonic probe of claim 8, wherein the controller generates a control signal corresponding to the sensed at least one RFID tag.

10. The wireless ultrasonic probe of claim 6, wherein the RFID antenna receives a RFID signal including identification information about an external device from a RFID antenna of the external device.

11. The wireless ultrasonic probe of claim 10, wherein the controller generates a control signal communicating with the external device corresponding to the received identification information.

12. The wireless ultrasonic probe of claim 11, wherein the wireless communicator transmits the echo-signal to the external device according to the control signal.

13. The wireless ultrasonic probe of claim 11, wherein the RFID antenna receives a RFID signal including setting information about the external device or an echo-signal previously stored in the external device from the external device according to the control signal.

14. The wireless ultrasonic probe of claim 1, further comprising:
a shielding film provided inside the housing to protect the inside of the housing from noise;
a printed circuit board (PCB) configured to provide a path of a signal inside the housing; and
a battery configured to supply power to the wireless ultrasonic probe,
wherein the RFID antenna is provided on at least one of the shield film, the PCB substrate, the battery and the inside of the housing.

15. A ultrasonic apparatus comprising:
a wireless ultrasonic probe configured to transmit ultrasonic waves at an object, receive echo-ultrasonic waves reflected from the object, and transmit an echo-signal corresponding to the received echo-ultrasonic waves in a wireless scheme; and
a main body configured to generate an ultrasonic image based on the echo signal received from the wireless ultrasonic probe,
wherein the wireless ultrasonic probe includes a RFID antenna provided inside a housing of the wireless ultrasonic probe to receive a RFID signal.

16. The ultrasonic apparatus of claim 15, wherein the RFID antenna includes:
a substrate including a first surface and a second surface being opposite and spaced away from the first surface; and
an antenna pattern formed on the first surface or the second surface.

17. The ultrasonic apparatus of claim 15, wherein the RFID antenna is provided such that a first direction perpendicular to the first surface and the second surface of the substrate is different from a second direction corresponding to an irradiation direction of the ultrasonic waves.

18. The ultrasonic apparatus of claim 17, wherein the RFID antenna is provided inside the housing such that an angle formed between the first direction and the second direction is 30 degrees or above.

19. The ultrasonic apparatus of claim 15, wherein the wireless ultrasonic probe further comprises a controller configured to generate a control signal to control the wireless ultrasonic probe.

20. The ultrasonic apparatus of claim 19, further comprising an input tag provided with a plurality of RFID that is selectable by a user.

21. The ultrasonic apparatus of claim 20, wherein the RFID antenna senses at least one RFID tag selected by the user in the input tag.

22. The ultrasonic apparatus of claim 21, wherein the controller generates a control signal corresponding to the sensed at least one RFID tag.

23. The ultrasonic apparatus of claim 19, wherein the RFID antenna receives a RFID signal including identification information about the main body from the main body.

24. The ultrasonic apparatus of claim 23, wherein the controller generates a control signal communicating with the main body corresponding to the received identification information.

25. The ultrasonic apparatus of claim 24, wherein the wireless ultrasonic probe transmits the echo-signal to the main body device according to the control signal.
